(19) United States
(12) Patent Application Publication
(10) Pub. No.: US 2017/0216179 A1
(43) Pub. Date: Aug. 3, 2017

(54) COSMETIC AND/OR DERMATOLOGICAL COMPOSITION FOR COLOURING THE SKIN

(71) Applicants: Cécile BIZE, Toulouse (FR); Michaël PAILLASSE, Toulouse (FR); Philippe DE MEDINA, Colomiers (FR); Sandrine SILVENTE-POIROT, L'Union (FR); Marc POIROT, L'Union (FR); Muriel BLANZAT, Toulouse (FR); Isabelle RICO-LATTES, Auzielle (FR); Emile PEREZ, Colomiers (FR); AFFICHEM, Toulouse (FR); CENTRE NATIONAL DE LA RECHERCHE SCIENTIFIQUE, Paris (FR); UNIVERSITÉ PAUL SABATIER - TOULOUSE III (UPS), Toulouse (FR)

(72) Inventors: Cécile BIZE, Toulouse (FR); Michaël PAILLASSE, Toulouse (FR); Philippe DE MEDINA, Colomiers (FR); Sandrine SILVENTE-POIROT, L'Union (FR); Marc POIROT, L'Union (FR); Muriel BLANZAT, Toulouse (FR); Isabelle RICO-LATTES, Auzielle (FR); Emile PEREZ, Colomiers (FR)

(73) Assignees: AFFICHEM, Toulouse (FR); CENTRE NATIONAL DE LA RECHERCHE SCIENTIFIQUE, Paris (FR); UNIVERSITÉ PAUL SABATIER - TOULOUSE III (UPS), Toulouse (FR); Cécile BIZE, Toulouse (FR); Michaël PAILLASSE, Toulouse (FR); Philippe DE MEDINA, Colomiers (FR); Sandrine SILVENTE-POIROT, L'Union (FR); Marc POIROT, L'Union (FR); Muriel BLANZAT, Toulouse (FR); Isabelle RICO-LATTES, Auzielle (FR); Emile PEREZ, Colomiers (FR)

(21) Appl. No.: 15/300,122

(22) PCT Filed: Mar. 24, 2015
(86) PCT No.: PCT/FR2015/000065

§ 371 Date: Sep. 28, 2016

(30) Foreign Application Priority Data
Mar. 28, 2014 (FR) 14 00746

(51) Int. Cl. A61K 8/63; A61K 8/73; A61K 8/34; A61Q 19/04; A61K 8/04

(52) U.S. Cl. CPC A61K 8/63; A61Q 19/04; A61K 8/042; A61K 8/345; A61K 8/731; A61K 2800/74

(57) ABSTRACT

The invention relates to a composition which contains an aqueous solution of certain elements of a family of sterol derivatives, which enable improved tanning of the skin due to considerable penetration of the molecules thereof. Preferably, the composition also contains an ionic solution of cationic association complexes constituting an acid amphiphilic carrier.

The invention relates to a cosmetic and/or dermatological composition to be used for colouring the skin.

The benefits of sunlight are well known: it boosts morale and promotes the synthesis of vitamin D. However, we also know that any unprotected sun exposure is a danger to the skin. That is why it has been ensured that it is no longer necessary to sunbathe or to conduct irradiation sessions with ultraviolet rays in appropriate cabins in order to obtain a tanned skin. Therefore, products have been proposed to preserve the “sun resistance” of the human body, i.e. the amount of radiation that it can withstand without irreversible damage.

To meet this demand of the cosmetics industry, many topical solutions of self-tanning products such as dihydroxyacetone (DHA) or erythrulose, have been proposed, which allow obtaining a tanned skin without exposure to harmful radiations. The coloration obtained results from the combination of the self-tanning product and the amino acids making up the proteins of the corneocytes of the stratum corneum of the skin according to the Maillard reaction (non-enzymatic reaction) inducing the formation of coloured products on the surface of the skin. These colorations appear after a few hours but disappear pretty quickly and are not generally evenly distributed on the skin.

Tanning by solar radiation generates, in the superficial layers of the skin, an increase of melanin, which is not the case with the current self-tanning products; the result is a significant difference of colorations obtained by the self-tanning products, even when homogeneous, from those obtained under the effect of sun radiation.

Moreover, it is known that tanning due to the increase of melanin in all the surface layers of the skin, reduces the impact of skin burns of subjects who have already suffered excessive exposure. It was determined that, under the effect of ultraviolet rays, keratinocytes secrete the αMSH hormone (melanocyte-stimulating hormone), which binds to melanocytes via receptors, and results in an increase in intracellular cyclic AMP and stimulates melanin production. Melanin is stored in vesicular structures called melanosomes, which mature in melanocytes and are transported in the multiple indentations of the melanocytes (see Nature Reviews, Molecular Cell Biology, Vol. 2, October 2001, p. 1-11). The melanosomes thus transported are transferred up to the skin keratinocytes, allowing for the migration of melanin in the successive layers of the skin up to the stratum corneum and therefore to skin tanning. Therefore, it appears that to increase skin tanning, it is best to stimulate the natural production of melanin and to act on the transfer of melanosomes from melanocytes to keratinocytes.

Regarding the first process mentioned above, it has already been proposed to apply, topically to the skin, a cosmetic milk rich in glycyrrhetinic acid. However, the effect of such a product is limited because melanogenesis stimulation is very poor, if any, in an in vitro model (Jung et al, Exp. Mol. Med. 2001, 33, 131). It was also proposed to increase the synthesis of melanin using a subcutaneous implant consisting of an analogue of the α-MSH hormone mentioned above, namely afamelanotide, but this implant implementation cannot be considered compatible with normal use in cosmetics: it is in fact a medical device, particularly useful for treating vitiligo.

Patent application WO 03/089449 describes novel derivatives of sterols; this patent application indicates that the new compounds it describes tend to redifferentiate tumour cells, which are dedifferentiated during their malignant transformation. Therefore, a person skilled in the art deduces that these compounds have an action that has no interest if one tries to act on a normal differentiated cell; therefore, he/she would not attempt to use them in cosmetics to obtain skin tanning with no malignant transformation, although it has been mentioned that some of these derivatives were efficient in stimulating melanin secretion in melanoma cells (de Medina et al. J. Med. Chem. 2009, 52, 7765). However, it has now been found, according to the invention, that if the compounds mentioned above were used in topically delivered compositions and despite the skin being a major obstacle to the penetration of active molecules, surprisingly a significant improvement of skin tanning would be obtained thanks to significant penetration of some molecules of patent application WO 03/089449, their stability after penetration and their photostability after sun exposure.

However, to improve skin penetration of these sterol derivatives, it was also proposed according to the invention, the implementation of a catanionic combination, according to patent application WO 2011/039379, in which the sterol derivative mentioned above is combined with a surfactant, the combination between the two molecules being obtained by an acid-base reaction and the active ingredient being an amphiphilic counter-ion having therapeutic properties. The carrier/active ingredient combination is bound by electrostatic interactions, allowing for an easy release of the active ingredient in tissues and spreading of the cosmetic effect by a delay related to the separation of the active ingredient. In other words, the carrier may play a triple role: facilitating the passage of the active component, having its own cosmetic effect on skin cells and having a delayed action for the release of the active ingredient. The carrier/active ingredient assembly allows for the combination of a positive ionization group of the active ingredient with the acid group of the carrier. Therefore, the active ingredient can be combined with the carrier by the R₃ substituent carried by carbon 6 of the cholesterol skeleton of the compound of formula (I) defined below.

Moreover, it should be highlighted that the compounds described in patent application WO 03/089449 have anticancer activity and that those of them which, according to the invention, may allow some tanning as indicated above, are present in skin areas subjected to sun exposure, and are, therefore, in place in irradiated sites where damage could occur, which would then allow them to play their anticancer role if such were the case.

Therefore, an object of the present invention relates to a topically applicable cosmetic composition to ensure skin tanning of a human subject, with or without irradiation under ultraviolet rays, characterized in that it contains, in a cosmetically acceptable vehicle, an aqueous solution of at least one compound of formula (I):

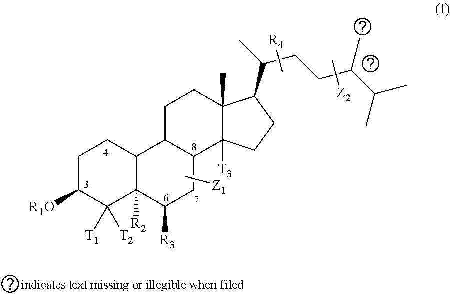

wherein

R₁ designates a hydrogen atom or R₁₀—CO, where R₁₀ represents H, CH₃ or C₂H₅,

R₂ represents hydrogen or OH,

R₃ represents a group X—(CH₂)ₙ—Y, where n is an integer from 1 to 4, X represents S, O or NH, and Y represents NH₂, imidazol-5-yl, indol-3-yl, piperidin-2-yl, piperidin-3-yl, piperidyn-4-yl, piperazin-2-yl, piperazin-3-yl, pyridin-2-yl, pyridin-3-yl or pyridin-4-yl,

R₄ represents hydrogen or OH in position 20, 22, 24, 25, 26 or 27 of the ring shown above to define formula (I), where OH is positioned so as to obtain an R or S asymmetric centre,

Z₁ and Z₂ represent the possibility of having single or double bonds between the C7 and C8 carbons, on the one hand, and the C22 and C23 carbons, on the other hand,

T₁, T₂, T₃ are, independently from one another, H or CH₃ in α or β, T₄ is H, CH₃ or C₂H₅ positioned to form an R or S asymmetric centre.

According to a first aspect of the invention, the composition contains a mixture of both compounds of formula (I) wherein R₁=R₄=T₁=T₂=T₃=H, R₂=OH, R₃=NH—(CH₂)₂-imidazol-5-yl and T₄=CH₃ or C₂H₅.

According to another aspect of the invention, the composition contains from 0.01 to 500 g/L of compound(s) of formula (I).

According to another aspect of the invention, the composition contains at least one tanning compound other than the compound(s) of formula (I).

According to another aspect of the invention, the tanning compound(s) it contains, independently from the compound(s) of formula (I), is dihydroxyacetone and/or erythrulose and/or at least one water-soluble dye.

According to another aspect of the invention, the composition contains at least one melanogenesis activator compound other than the compound(s) of formula (I) selected from the group made up of substrates for melanin biosynthesis and melanogenesis biological activators able to act by stimulating the synthesis of melanin and/or the transfer of melanosomes from melanocytes to keratinocytes.

According to another aspect of the invention, when the composition contains one (or more) melanogenesis activator(s) independently of the compound(s) of formula (I), said activator(s) is (or are) chosen from the group made up of L-tyrosine or derivatives thereof such as N-acetyl-L-tyrosine, L-dihydrophenylalanine, aliphatic diols such as propylene glycol, cyclic diols, adenosine-1 receptor agonists, adenosine-2 receptor agonists, α-hydroxyacids and derivatives thereof, β-hydroxyacids and derivatives thereof, retinoids and derivatives thereof, pro-opiomelanocortic peptides, α-MSH or its analogs, MC1R receptor agonists, cAMP analogs, psoralens, PAR-2 receptor activity activators.

According to another aspect of the invention, the composition is applied to the skin in an amount between 0.0001 and 100 mg/cm²/day of compound(s) of formula (I).

As previously stated, the invention also provides improvement of the transport of the compound(s) of formula (I) to the skin keratinocytes by combining this (these) compound(s) with at least one acidic amphiphilic carrier (T), to form catanionic mixture complexes by electrostatic interactions.

According to another aspect of the invention, the composition, therefore, contains an ionic solution of catanionic mixture complexes, each mixture is formed by a molecule of formula (I) and one or two molecules of an amphiphilic acid carrier (T).

According to another aspect of the invention, a catanionic mixture is obtained by at least one acid-base reaction carried out between the acid group of a carrier (T) and an amino group of substituent R₃ of carbon in position 6 of the compound of formula (I).

According to another aspect of the invention, the carrier (T) is a bolaform surfactant derived from sugar comprising an acid function within its structure.

According to another aspect of the invention, the carrier (T) acid function is selected from the group made up of carboxylic acid COOH, sulfuric acid O—SO₃H, sulfonic acid SO₃H, phosphoric acid O—P(O) (R₅O)OH, wherein R₅ is a C₁-C₆ alkyl group, phosphonic acid O—P(O)R₆OH and phosphinic acid P(O)R₆OH, wherein R₆ is H or a C₁-C₆ alkyl group.

According to another aspect of the invention, the sugar derivative is a monosaccharide or polysaccharide derivative.

According to another aspect of the invention, the sugar derivative contained in the composition is a glucose or lactose derivative.

According to another aspect of the invention, the sugar acid derivative comprises a sugar structure on which a linear or branched aliphatic chain (CH₂)ₚ is bonded by a NH group of which the end opposite to the sugar structure carries an acid group, p being an integer having a value from 4 to 10.

According to another aspect of the invention, the ratio of the weight amounts A of compound(s) of formula (I) and B of carrier(s) (T) is between 0.001 and 1000.

According to another aspect of the invention, the composition contains from 0.01 to 150 g of the totality of components A and B per litre of composition.

According to another aspect of the invention, the composition comprises at least one adjuvant selected from the group made up of at least one thickener and/or water-soluble dye and/or fragrance and/or alcohol and/or vitamin and/or emulsifier, used alone or in combination with optionally a co-emulsifier and/or oil or another fat and/or preservative and/or antioxidant and/or bactericide.

According to another aspect of the invention, the composition contains at least one chemical or physical sun filter, or a combination of such filters.

According to another aspect of the invention, the composition is packaged as nanocapsules or nanosized catanionic vesicles suspended in a liquid medium.

According to another aspect of the invention, the composition is packaged in the form of hydrogels, creams, lotions, emulsions or aerosols.

To better understand the aim of the invention, several embodiments will be described now as purely illustrative and non-limitative examples. The results of the examples detailed below have been shown in the drawings.

In the drawings:

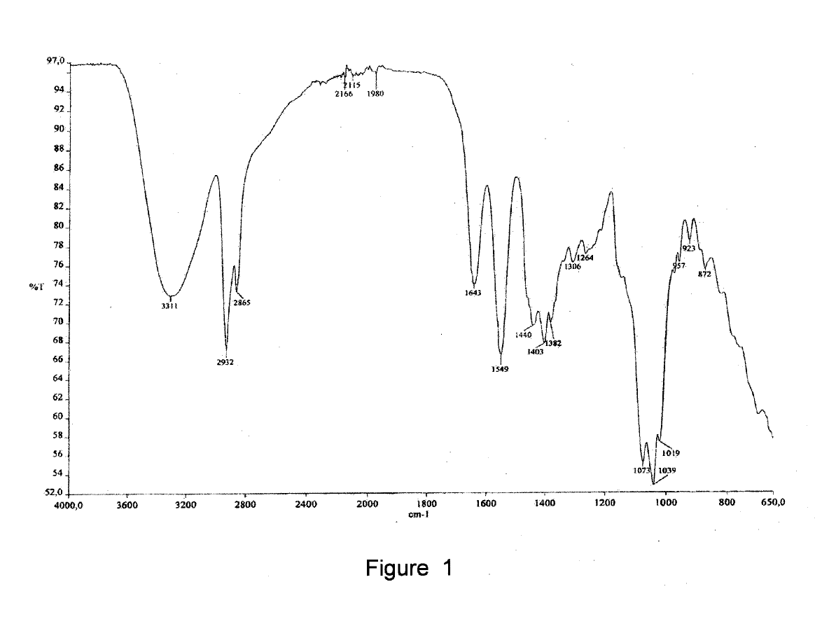

FIG. 1 shows the IR spectrum for the product obtained in Example 3;

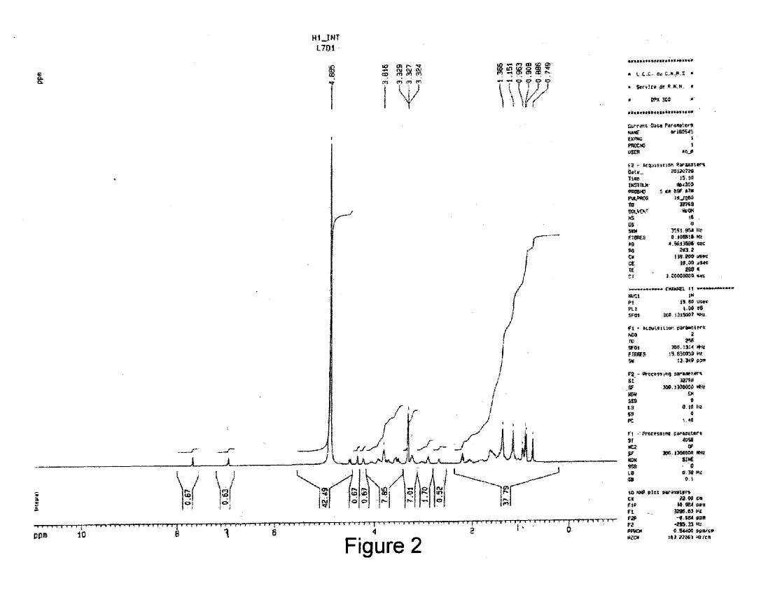

FIG. 2 shows the NMR spectrum for the product obtained in Example 3;

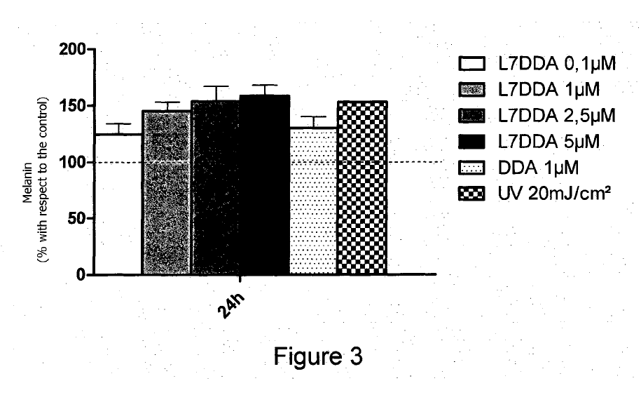

FIG. 3 shows the results obtained in Example 5;

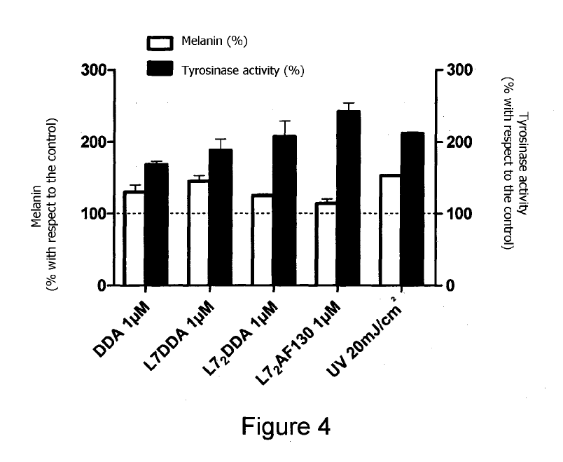

FIG. 4 shows the results obtained in Example 6;

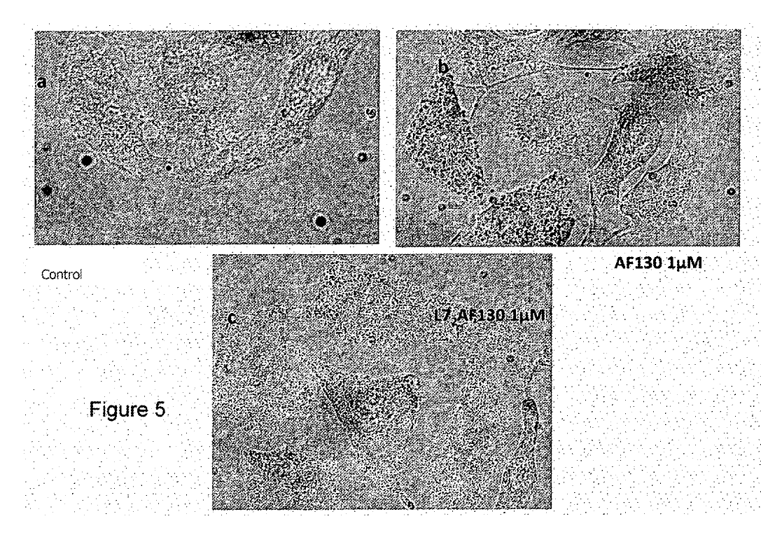

FIGS. 5a, 5b, 5c show a visualization of melanosomes in cells treated according to Example 7;

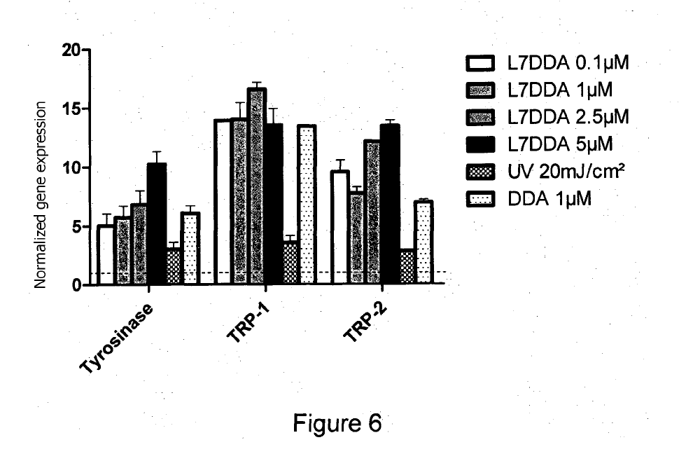

FIG. 6 shows the result of treatment according to Example 8 for gene expression in treated cells;

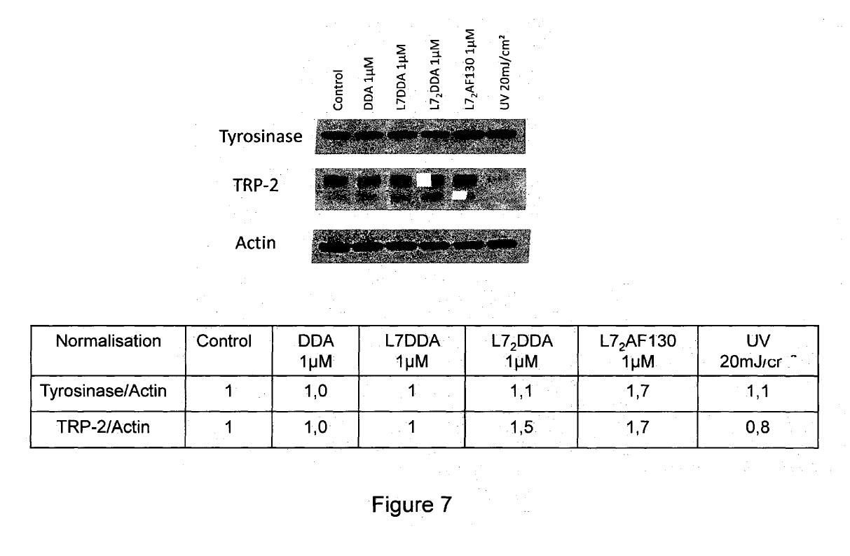

FIG. 7 shows the result of treatment according to Example 9 in modulating proteins, this result being visualized by “Western Blot” technique;

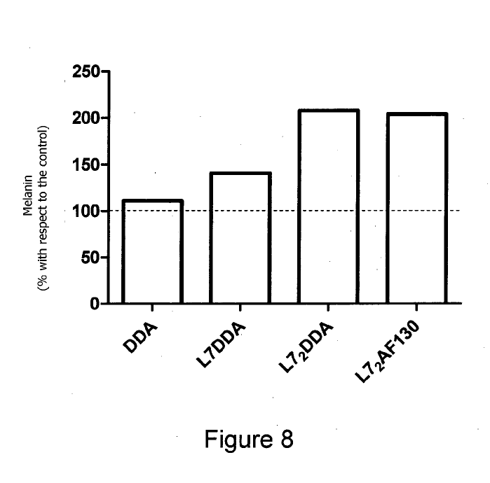

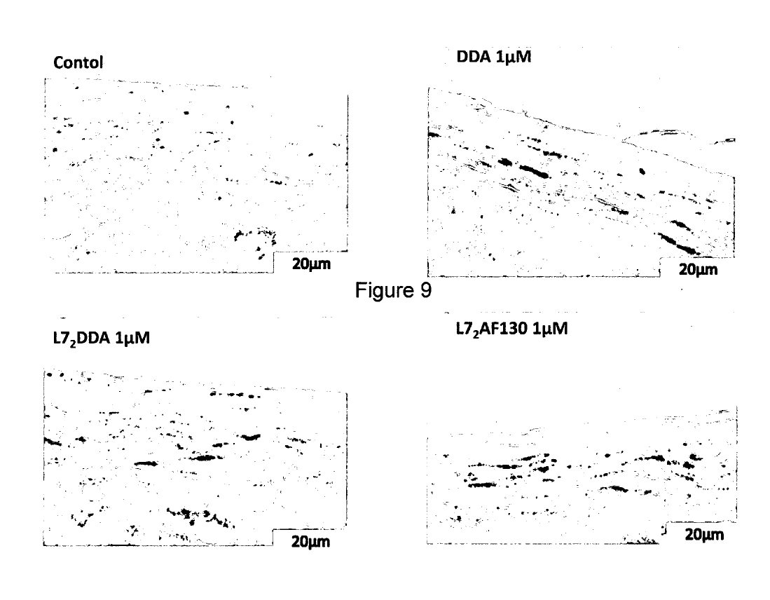

FIGS. 8 and 9 show the result of treatment with compositions in accordance with the invention on reconstituted skin;

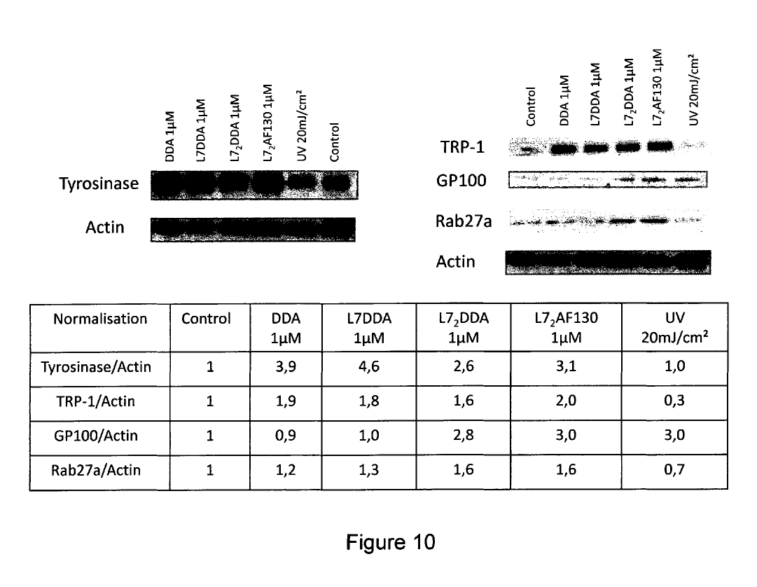

FIG. 10 shows the result of treatment according to Example 10 in modulating proteins involved in melanogenesis;

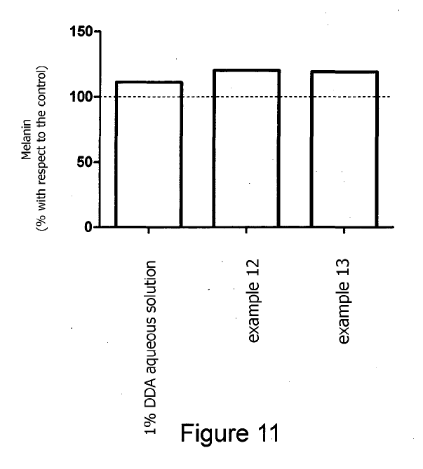

FIG. 11 shows the quantities of melanin synthesized on a reconstituted skin sample after application of a composition according to Examples 12 and 13, wherein said quantities and skin samples are defined as described in Example 10.

### EXAMPLE 1: SYNTHESIS OF THE COMPOUND OF FORMULA (I) WHEREIN T₄=H AND Z₁=Z₂=SINGLE BOND

The process described below is extracted from the published patent application WO03/089449. The compound (hereinafter referred to as DDA) corresponds to the systematic name: 5α-hydroxy-6β-[2-(1H-imidazol-4-yl)ethylamino]cholestan-3β-ol.
a) Preparation of 5,6-α-epoxycholestan-3β-ol:

Meta-chloro-peroxybenzoic acid (0.73 g, 4.25 mmol, purity of 70-75% by weight) was dissolved in methylene chloride (10 ml) and added dropwise, under stirring, to a mixture of cholesterol (1 g, 2.5 mmol) dissolved in methylene chloride (25 ml). Stirring was maintained overnight. The reaction mixture was washed with aqueous sodium sulfite solution (10% by weight) and sodium hydrogen carbonate (5% by weight) and a saturated solution of a mixture of sodium chloride and potassium chloride. The organic phase was dried over anhydrous magnesium sulfate. Vacuum evaporation of the organic solvent gave 0.7 g of white needles (70% yield).

The proportion of α and β epoxide isomers was determined by proton NMR: 78% of α-epoxide, 22% of β-epoxide were found.

Characterization of the product: ¹H NMR (200 MHz, MeOD) δ ppm 2.89 (d, 1H, J=4.37 Hz, H-6); 3.04 (d, J=2.43 Hz, H-6) 3.91 (m, 1H, H-3); MS DCI/NH₃MH⁺ 403] DCI/MNH₄⁺, m/z: 403 [MNH₄]⁺. The α and β isomers were separated by liquid chromatography on silica (toluene/ethyl ether 85/15). The α isomer had a melting point Mp=141-142° C.; the β-isomer had a melting point Mp=131-132° C.

To complete characterization, thin layer chromatography was performed (ethyl acetate); we obtained: Rf=0.69 (brown colour after developing with a mixture of sulfuric acid-methanol).

b) Synthesis of the amino sterol from the α-epoxy-sterol obtained in step a)

Lithium perchlorate (0.75 mmol) and an amine in its basic form (1 mmol), namely, 2-(1H-imidazol-4-yl)-ethylamine, were dissolved in anhydrous ethanol (1 ml). This solution was added under an argon stream to an ethanol solution (3 ml) of the α-epoxy-sterol obtained according to a) above (100 mg, 0.25 mmol). The reaction mixture was stirred and refluxed for 6 days. Progress of the reaction was monitored by thin layer chromatography (TLC). The solvent was removed by evaporation and the residue was washed with ethyl ether (5×3 ml) and hexane (5×20 ml). The residue was dissolved in water and acidified with 2M HCl (2 ml).

The solution was pre-purified on a grafted silica cartridge (“sep-pack cartridge C18 RP”, 500 mg, Waters); excess polyamine was removed by flushing water on the cartridge (5 ml); the product was eluted with 1/1 CH₃CN/H₂O (5 ml). The product was purified by reverse phase HPLC using a linear gradient of a starting mixture of 95/5/0.1 H₂O/CH₃CN/TFA to 95/5/0.1 CH₃CN/H₂O/TFA achieved over 60 minutes (flow rate=1 ml/min; X=210 nm). The fraction of interest was purified again under isocratic conditions using a mobile phase consisting of a mixture of 44% of (95/5/0.1 CH₃CN/H₂O/TFA) 56% of (95/5/0.1 H₂O/CH₃CN/TFA) (flow rate=1 ml/min; λ=210 nm).

The product obtained was first characterized by TLC (methanol). Then high performance chromatography (HPLC) was performed on a “Perkin-Elmer LC200 Series” device equipped with “Ultrasep ES100RP10” column (6 μm particles), 250 mm length and 8 mm diameter, manufactured by “Bischoff”.

HPLC profile: 44% B #=220 nm rt=44-50 min

Characterization of the product: ESI-MS, m/z 514.5 [M+H]⁺.

### EXAMPLE 2: SYNTHESIS OF COMPOUNDS OF FORMULA (I) WHEREIN T₄=CH₃/C₂H₅ (70/30 MIXTURE) AND Z₁=Z₂=SINGLE BOND

A mixture of sitosterol and campesterol (70/30 by weight) was used as sterol, in step a) of Example 1. For the reaction on the obtained α-epoxysterol, the same amine as in Example 1 step b) was used.

A mixture of the 2 following compounds was obtained:

5α-hydroxy-6β-[2-(1H-imidazol-4-yl)ethylamino]sitostan-3β-ol and

5α-hydroxy-6β-[2-(1H-imidazol-4-yl)ethylamino]campestan-3β-ol.

The product obtained (hereinafter referred to as “AF130”) was characterized by TLC (ethyl acetate): Rf=0.69

Characterization of the product:

ESI-HRMS m/z: 528.8323 [M+H]+(Δm=0.3 mDa) (campesterol derivative) and 524.8590 [M+H]+(Δm=0.4 mDa) (sitosterol derivative)

### EXAMPLE 3

a) Preparation of the amphiphilic carrier used to prepare the catanionic mixture complex implemented in the subsequent examples. Lactobionic acid was directly reacted with 8-aminooctanoic acid (see Patent Publication WO 2011/039379).

1) Synthesis of sodium 8-lactobionamidooctanoate

To a solution of sodium hydroxide (300 mg, 5.59 mmol) in 50 mL of methanol were successively added 8-aminooctanoic acid (888 mg, 5.59 mmol) and lactobionic acid (2.00 g, 5.59 mmol). The mixture was stirred at 50° C. for 24 h; the solvent was then evaporated under reduced pressure; the residue obtained was purified by chromatography column on silica gel (eluent: CHCl₃/CH₃OH/H₂O: 2.5/7/0.5, Rf=0.8). A white solid was collected at a yield of 56%.

Characterization of the product:

IR(neat) cm⁻¹: 1643 (v_{c=o} (amide)st);

1547 (v_{c=o} (carboxylate) asymmetric);

1405 (v_{c=o} (carboxylate) symmetric)

ESI-MS, m/z: 498 [M+Na]⁺.

The product obtained has the following formula:

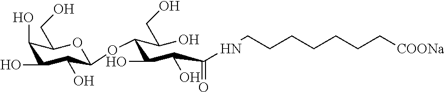

2) Synthesis of 8-lactobionamidooctanoic acid

To a solution of sodium 8-lactobionamidooctanoate (1.00 g, 2 mmol) in 30 mL of H₂O was added approximately 2 g of proton exchange resin (Dowex 50WX8). The reaction mixture was stirred for 2 h at room temperature, then filtered and concentrated. The residue obtained was purified by column chromatography on silica gel (eluent: acetone/H₂O: 9/1; Rf: 0.5). A white solid was collected at a yield of 57%.

Characterization of the product:

¹H NMR (300 MHz, MeOD) δ ppm: 1.05-2.15 (m, 10H, CH_{2β}, CH_{2γ}, CH_{2δ}, CH_{2ε}, CH_{2ζ}); 2.17 (t, 2H, CH_{2α}); 3.12 (m, 2H, CH_{2η}); 3.46-4.12 (m, 10H, CH and CH₂ of the sugar unit); 4.28 (m, 1H, H) 4.30 (m, 1H, H alpha of CONH); 4.54 (d, J=9 Hz, 1′H, anomeric H)

IR(neat) cm⁻¹: 1645 (v_{c=o} (amide) st);

1726 (v_{c=o} (acid) st)

ESI-HRMS, m/z: 522.2162 [M+Na]⁺ (Δm=0.1 mDa).

The product obtained is called L7 and is represented by the following formula:

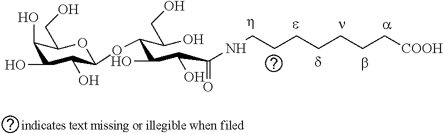

b) Preparation of a catanionic mixture complex between the amphiphilic carrier of step a2) of this example and the product of Example 1.

To a solution of 8-lactobionamidooctanoic acid (330 mg 0.66 mmol) in 30 mL of deionized H₂O was added 339 mg of the product of Example 1 (0.66 mmol). The reaction mixture was then stirred at room temperature for 24 h. This gave a homogeneous solution which, after concentration, quantitatively gave the product represented by the below formula:

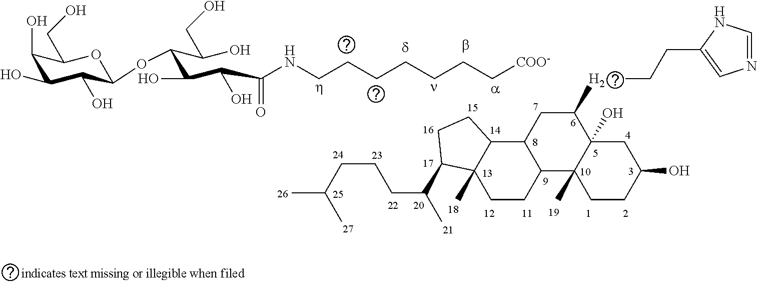

The resulting product is a pale yellow powder; it is hereinafter referred to as L7DDA as it consists of a L7 molecule combined with a DDA molecule.

Characterization of the product obtained:

¹H NMR (300 MHz, MeOD) 5 ppm: 0.75 (s, 3H, CH₃₍₁₉₎); 0.89 (m, 3H, CH₃); 0.91 (m, 3H, CH₃); 1.05-2.15 (m, 45H, CH_{2β}, CH_{2γ}, CH_{2δ}, CH_{2ε}, CH_{2ζ}, 35H for CH and CH₂ of DDA); 2.21 (t, 2H, CH_{2α}); 3.14 (m, 2H, CH_{2η}); 3.42-4.10 (m, 12H, CH and CH₂ of the sugar unit, +CH₃ and CH₆); 4.21 (m, 1H, H); 4.34 (m, 1H, H alpha to CONH); 4.50 (d, J=9 Hz, 1H, anomeric H); 6.92 (s, 1H, C=CH(NH); 7.80 (s, 1H, HN—CH=N).

The NMR spectrum is provided in FIG. 2; it shows the formation of the ion pair which combines an L7 molecule and a DDA molecule. The chemical shift of CH₂ group alpha to a carboxylic acid moiety COOH is different from the group alpha to a carboxylate group COO⁻; in this example, CH₂—COOH has a chemical shift of 2.17 ppm while CH₂—COO⁻ has a chemical shift of 2.21 ppm.

IR(neat) cm⁻¹: 1643 (v_{c=o} (amide) st); 1549 (v_{c=o} (asymmetric carboxylate)); 1403 (v_{c=o} (symmetric carboxylate)).

The IR spectrum, provided in FIG. 1, shows that step b) causes the disappearance of the carboxylic acid function characteristic band between 1800 and 1650 cm⁻¹ in favour of two new bands in the 1610-1550 cm⁻¹ region (asymmetric elongation of COO⁻ (carboxylate group)) and 1450-1400 cm⁻¹ (symmetric elongation of COO⁻ (carboxylate group)). Thus, in FIG. 1, the disappearance of the characteristic band of the carboxylic acid function characteristic band at 1726 cm⁻¹ and the appearance of symmetrical and asymmetrical elongation bands of the carboxylate group at 1549 cm⁻¹ and 1403 cm⁻¹, respectively can be observed.

CAC (Critical Aggregation Concentration) (aqueous solution, 25° C.): 2.6×10⁻⁵M.

### EXAMPLE 4

The same process as for step b) of Example 3 can be used to form a combination complex between an amphiphilic acid carrier according to Example 3a2) and any compounds of formula (I) as all these compounds comprise at least one NH group allowing bonding with the carrier acid function. In particular, a complex mixture was obtained by combining the product of Example 2 with that of Example 3a2): this mixture was hereinafter named L7AF130.

The carrier/active component assembly allows to combine a positive ionization group of the active component with the acid group of the carrier. Therefore, the active component can be combined to the carrier by the R₃ substituent carried by carbon 6 of the compound of formula (I). In the case of AF130 or DDA active components, we can combine two L7 carrier molecules on two amino functions of the R₃ substituent. From the process for obtaining Example 3b, mix in water at room temperature, two equivalents of L7 molecule for one equivalent of active component, as described for example 3b. Once again, the mixture was initially a suspension but became a clear solution, indicating that the resulting complex, hereinafter named (L7)₂(AF130) or (L7)₂(DDA) depending on the active component, was water soluble.

### EXAMPLE 5: STIMULATION OF THE SYNTHESIS OF MELANIN WITH A COMPOUND OF FORMULA (I) NAMED DDA AND A COMBINATION COMPLEX ACCORDING TO EXAMPLE 3A2)

Quantification of synthesized melanin by healthy murine melanocytes was performed according to the protocol described by Ando et al. (J. Lipid Res., 1999, 40, 1312).

Cells were seeded and treated for 24 h with the test product or the used vehicle (ethanol) and with, as a positive control, exposure to a dose of 20 mJ/cm² of ultraviolet radiation.

5 Million cells were isolated after centrifugation at 1500 rpm for 5 min at 4° C. The pellet was washed twice with PBS and then transferred into an Eppendorf tube and centrifuged at 5000 g for 5 min. The supernatant was removed. 200 μL of water and 1 mL of EtOH/ether (1/1) were added (melanin was insoluble in this mixture). After 15 min at room temperature, the totality was again centrifuged at 5000 g for 5 min; the supernatant was removed. The pellet was then taken up with 300 μL of a solution of NaOH(1M) in H₂O/DMSO 90/10 and placed at 80° C. for one hour. Absorbance was measured at 470 nm. The amount of synthesized melanin was expressed based on the control (=100%).

The results are shown in FIG. 3. It shows that the addition of a DDA compound of formula (I), with or without carrier, results in an increase of the synthesis of melanin in the investigated cells.

### EXAMPLE 6

### Study of the Synthesis of Melanin by Studying the Stimulation of Tyrosinase Activity

The study was conducted on healthy murine melanocytes. The results were compared with those obtained with a positive control subjected to a dose of 20 mJ/cm² of ultraviolet radiation. The cells were treated for 24 h with either the vehicle used (ethanol) or with the test product. The tyrosinase activity was quantified according to the protocol described by Ando et al. (J. Lipid Res. 1999, 40, 1312) as explained below.

5 Million cells were isolated after centrifugation at 1500 rpm for 5 min at 4° C. The pellet was washed twice with PBS and then transferred into an Eppendorf tube and centrifuged at 5000 g for 5 min. The supernatant was removed. 1 mL of 0.5% by mass sodium deoxycholate solution was added (cell lysis). After 15 min at 0° C., 3 mL of a solution of 0.1% by mass of L-DOPA in 0.1M phosphate buffer (pH 6.8) were added. The whole was placed at 37° C. for 10 min. Tyrosinase activity was spectrophotometrically analysed by following the oxidation of L-DOPA to DOPAchrome. Absorbance was measured at 475 nm. The tyrosinase activity was expressed based on the control (=100%), which determines the cell basal tyrosinase activity.

The results are illustrated in FIG. 4. It shows that the tyrosinase activity in cells increased when cells were treated with a composition containing the DDA active component, with or without L7 carrier or containing AF130 active component combined, for each molecule, with two molecules of L7 carrier, with respect to the control. It also shows that an increase in tyrosinase activity is associated with an increase in melanin synthesis in the cell.

### EXAMPLE 7: STUDY OF THE SYNTHESIS OF MELANIN BY MELANOSOME VISUALIZATION

Healthy murine melanocytes were treated as described in Example 6 by using untreated cells or cells treated with 1 μM of AF130 or 1 μM of (L7)₂AF130. The melanin synthesis was visualized by staining according to the DAPI technique: the nuclei of cells fluoresce in blue and, by contrast, melanosomes are highlighted, which are visualized in black. FIG. 5a corresponds to the visualization of untreated cells, FIG. 5b corresponds to the visualization of cells treated with 1 μM of AF130 and FIG. 5c corresponds to the visualization of cells treated with 1 μM of (L7)₂AF130: it is observed that the quantity of melanosomes and, therefore, melanin synthesis is greatly enhanced by treatment according to example 6 (comparison with FIG. 5a).

The DAPI technique is performed as follows: cells are seeded on previously sterilized slides by washing with ethanol; they are treated with the studied active molecule or with a vehicle (ethanol).

After 24 hours of treatment, cells were fixed using a formaldehyde solution diluted to one tenth (3.7% by weight) in PBS for 15 min at room temperature. Formaldehyde was then removed and the cells were washed with PBS. 10 μl of a DAPI solution to 1/500^{th} in Mowiol (mounting solution) were deposited on a slide. The cover slip was placed on the drop, cells being present between the slide and the cover slip. The slides were placed at 4° C. overnight before being observed. For each sample, an image in “DAPI” mode was performed, only allowing visualization of nuclei as well as the same image in “visible” mode. The two images were then superimposed.

### EXAMPLE 8

This example shows in healthy murine melanocytes, that treatment with compositions according to the invention allows to increase the expression of genes corresponding to tyrosinase and TRP-1 and TRP-2 enzymes (TRP=Tyrosinase Related Protein), which are involved in melanogenesis.

Cells were seeded and treated for 24 h with the molecules the action of which we want to study, that is to say here, with DDA (1 μM), on the one hand, and with L7DDA (0.1 or 1 or 2.5 or 5 μM) on the other. The results were compared with irradiated ultraviolet cells as in Example 6. FIG. 6 shows, as ordinate, the expression of genes corresponding to tyrosinase, TRP-1 and TRP-2.

To quantify the expression of these genes, RNA extraction from cells was performed. Melanocytes were treated for 24 h with the active molecule or the control agent. 1 mL of Trizol® was added; after 5 min at room temperature, the totality was placed in an Eppendorf tube. 200 μL of CHCl₃ were added at 4° C. The Eppendorf tubes were stirred manually, by turning them during 15 sec, left for 5 min at room temperature and then centrifuged at 16000 g at 4° C. for 10 min. The colourless aqueous phase was isolated. 500 μL of isopropanol (−20° C.) were added. The Eppendorf tubes were stirred manually, by turning them during 15 sec, left for 15 min at room temperature and then centrifuged at 16200 g at 4° C. for 10 min. The supernatant was removed. The pellet was washed with 1 mL of 75% ethanol (−20° C.). After centrifugation at 16000 g at 4° C. for 5 min, the supernatant was removed. 10 min after drying, the pellet was resuspended with 30 μL of RNAase-free water. The quantity and purity of RNA were estimated by spectrophotometry (230, 260 and 280 nm).

A RT-qPCR was performed; in an Eppendorf tube, were added: 1 μL of reverse transcriptase, 4 μL of 5× reaction mixture “iScript®5×”, the amount of RNAase-free water needed to reach a total volume of 20 μL and the corresponding volume for 1 μg of RNA. The Eppendorf tubes were then vortexed, centrifuged for a few seconds and then placed in the thermocycler (program: 5 min at 25° C., then 30 min at 42° C., then 5 minutes at 85° C. and then back to 4° C.)

An amplification was then performed. The reference genes were: glyceraldehyde 3-phosphate dehydrogenase and Cyclophilin A1. Genes of interest were: Tyrosinase, TRP-1 and TRP-2. For each gene (reference or of interest), a mixture was prepared with RNAase-free water, Sybr Green® and the corresponding primer.

The resulting cDNAs were taken up with 180 μL of RNAase-free water. 5 μL of cDNA were added at the bottom of each well. 20 μL of mixture were added on the edge of each well. The plate was then covered with an adhesive plastic and centrifuged at 4000 rpm until no more bubbles were observed.

The plate was then placed in the thermal cycler: 95° C. for 3 min, followed by 50 amplification cycles of 15 sec at 95° C. and one minute at 60° C. At the end of these 50 cycles, the melting curves were generated at 95° C. for 1 min and 95° C. for 10 sec (80 cycles). One can thus refer, in FIG. 6, to the stimulation of gene expression for the different tested products. The digital results are provided in the table below.

| | ΔΔCT | ΔΔCT | ΔΔCT |
|---|---|---|---|
| | (tyrosinase) | (TRP-1) | (TRP-2) |
| L7DDA 0.1 μM | 4.98 | 13.93 | 9.57 |
| L7DDA 1 μM | 5.76 | 14.00 | 7.75 |
| L7DDA 2.5 μM | 6.83 | 16.58 | 12.13 |
| L7DDA 5 μM | 10.25 | 13.53 | 13.47 |
| DDA 1 μM | 6.09 | 13.45 | 6.97 |
| UV 20 mJ/cm² | 2.99 | 3.54 | 2.83 |

It was found that the treatment of cells with DDA and L7DDA products provided results showing that the enzymes involved in melanogenesis are stimulated as much as with UV irradiation.

### EXAMPLE 9: EFFECT ON THE MODULATION OF PROTEINS INVOLVED IN MELANOGENESIS (WESTERN BLOT)

In this example, the test treatment was carried out on healthy murine melanocytes. Treatments were performed with 1 μM of the DDA compound and also with 1 μM of L7DDA and (L7)₂DDA and (L7)₂AF130 compounds. The positive control was carried out with cells that had received a dose of 20 mJ/cm² of UV radiation. The reference protein was actin (43KDa). After 24 hours of treatment, cells were scraped into cold PBS (4° C.) and then centrifuged at 1500 rpm at 4° C. for 5 min. The supernatant was removed.

The pellet was taken up with 100 μL of lysis buffer (0.1M of TRIS-HCl, pH=7.4, 1% Igepal, 0.01% SDS, to which mixture were added protease inhibitors (1% by volume of the mixture)) and vortexed for several seconds. After 30 min at 4° C., the mixture was vortexed again and subjected to ultrasound for 5 sec. The Eppendorf tubes were finally centrifuged at 10000 rpm at 4° C. for 10 min. The supernatant (=protein extract) was transferred to a new Eppendorf tube. A Bradford assay was performed to quantify the proteins.

Based on the results of this assay, samples were prepared for deposit on SDS-PAGE gel (samples prepared at 1 μg/μL): 10 μL, i.e. 10 μg, of proteins were deposited for each sample.

After gel transfer in liquid phase of the proteins on a polyvinylidene fluoride membrane, membranes were incubated with the primary antibodies of interest (Tyrosinase: 1/2000^{th}, Actin: 1/10000^{th}, TRP-2: 1/2000^{th}) and then with the secondary antibody directed against the gene of interest (in all cases, at 1/1000^{th}), coupled with HRP (horseradish peroxidase). Finally, after an incubation of 5 min with 1 ml of ECL (enhanced chemiluminescence)/membrane, the membranes were developed in a darkroom. This information was also used to perform the dosages as described in Example 11 below (results in FIG. 10).

The result of the Western Blot thus preformed is shown in FIG. 7. It is observed that the treatment of cells with compositions according to the invention generates a tyrosinase production stimulation similar to that obtained with ultraviolet irradiation; stimulation is stronger for the treatment with (L7)₂AF130. This result confirms the one described in Example 8.

### EXAMPLE 10: TREATMENT OF RECONSTITUTED SKIN

In this example, test treatment was carried out on “RHE/MEL light tanned” reconstituted skin epidermis marketed by the “StratiCELL®” company. The medium of the epidermis was changed every day before the test treatment. The treatment consists of adding 1 μM of the treatment product in the medium, twice a day for five days. The epidermi were isolated and placed in an Eppendorf tube with 400 μL of “Solvable” reagent (supplied by Perkin-Elmer) and then heated at 100° C. for 1 h. Absorbance was measured at 490 nm. The amount of melanin synthesized was expressed based on the control (=100%). The results are shown in FIG. 8. The epidermis tanning at the end of treatment were studied by histology highlighting the evidence of melanin deposits in treated skins. At the end of treatment, tissues were placed in a 4% formaldehyde solution and embedded in paraffin and cut. A Fontana-Masson staining (argentaffine method based on silver nitrate) highlights melanin deposits in black. Cut observation through an optical microscope provides the images of FIG. 9.

### EXAMPLE 11: STUDY OF THE MODULATION OF DIFFERENT PROTEINS INVOLVED IN MELANOGENESIS ON A RECONSTITUTED SKIN MODEL

“RHE/MEL light tanned” reconstituted skin samples marketed by the “StratiCELL®” company were used. The sample was treated according to the procedure of Example 9 with the same UV positive control and the same control with actin. The sample was treated twice a day by action of 1 μM of DDA, L7DDA, (L7)₂DDA and (L7)₂AF130 products.

Details of the method used have already been provided in Example 9 (GP100: 1/500^{th}, TRP-1 1/1000^{th}, Rab27a: 1/500^{th}).

The results are shown in FIG. 10 and show that the tyrosinase and proteins involved in melanogenesis TRP-1 and GP100 (key protein of melanosomes biogenesis) are more highly stimulated by the treatment with a composition according to the invention than by UV exposure. Moreover, Rab27a, a protein involved in the transfer of melanosomes from melanocytes to keratinocytes, is more strongly stimulated by treatment with catanionic combinations.

Examples 12-22 describe compositions according to the invention used for application on human reconstituted skin such as those used in Examples 10 and 11. The compounds are, depending on the case, described with their chemical names or INCI names (International Nomenclature of Cosmetic Ingredients) and the quantities indicated are percentages by weight (percentage of active material for the DDA and catanionic mixtures). Models of reconstituted skin were treated twice a day for three days with 2 mg/cm² of the formulations described below (Examples 12-22). The results are illustrated in FIG. 11.

### EXAMPLE 12: THE COMPOSITION, WHOSE FORMULATION IS GIVEN BELOW, IS A HYDROALCOHOLIC GEL PREPARED AS FOLLOWS: WATER AND ETHANOL WERE FIRST MIXED, HYDROXYPROPYL CELLULOSE WAS ADDED, STIRRING WAS PERFORMED UNTIL COMPLETE DISSOLUTION THEN THE OTHER COMPONENTS WERE ADDED WHILE STIRRING

| | |
|---|---|
| Ethanol | 50 |
| Glycerin (sold under the name “Pricerine 9091 ®” by the | 5 |
| company “Croda”) | |
| Hydroxypropyl cellulose (sold under the name “Klucel H | 1 |
| CS ®” by the company “IMCD” | |
| DDA dilactate Powder (as active molecule) | 1 |
| Water | up to 100 |

### EXAMPLE 13: THE COMPOSITION, WHOSE FORMULATION IS GIVEN BELOW, IS A HYDROALCOHOLIC GEL PREPARED AND USED AS IN EXAMPLE 12

| | |
|---|---|
| Ethanol | 50 |
| Glycerin (sold under the name “Pricerine 9091 ®” by the | 5 |
| company the company “Croda”) | |
| Hydroxypropyl cellulose (sold under the name “Klucel H | 1 |
| CS ®” by the company “IMCD” | |
| (L7)₂(DDA) Powder (as active molecule) | 1 |
| Water | up to 100 |

### EXAMPLE 14: THE COMPOSITION, WHOSE FORMULATION IS GIVEN BELOW, IS A HYDROGEL PREPARED AS IN EXAMPLE 12 WITHOUT ETHANOL, WHICH WAS ABSENT IN THE FORMULATION

| | |
|---|---|
| Hydroxyethyl cellulose (sold under the name “Natrosol | 1.5 |
| 250 HR ®” by the company “IMCD”) | |
| Glycerin (sold under the name “Pricerine 9091 ®” by the | 5 |
| company “Croda”) | |
| DDA dilactate Powder (as active molecule) | 1 |
| Water | up to 100 |

### EXAMPLE 15: THE COMPOSITION, WHOSE FORMULATION IS GIVEN BELOW, IS A HYDROGEL PREPARED AS IN EXAMPLE 14

| | |
|---|---|
| Hydroxyethyl cellulose (sold under the name “Natrosol | 1.5 |
| 250 HR ®” by the company “IMCD”) | |
| Glycerin (sold under the name “Pricerine 9091 ®” by the | 5 |
| company “Croda”) | |
| Propylene glycol | 5 |
| DDA dilactate Powder (as active molecule) | 1 |
| Water | up to 100 |

### EXAMPLE 16: THE COMPOSITION, WHOSE FORMULATION IS GIVEN ABOVE IS A HYDROGEL PREPARED AS IN EXAMPLE 14

| | |
|---|---|
| Hydroxyethyl cellulose (sold under the name “Natrosol | 1.5 |
| 250 HR ®” by the company “IMCD”) | |
| Glycerin (sold under the name “Pricerine 9091 ®” by the | 5 |
| company “Croda”) | |
| Propylene glycol | 5 |
| L7DDA Powder (as active molecule) | 1 |
| Water | up to 100 |

### EXAMPLE 17: THE COMPOSITION, WHOSE FORMULATION IS GIVEN BELOW, IS A HYDROGEL PREPARED AS IN EXAMPLE 14

| | |
|---|---|
| Hydroxyethyl cellulose (sold under the name “Natrosol | 1.5 |
| 250 HR ®” by the company “IMCD”) | |
| Glycerin (sold under the name “Pricerine 9091 ®” by the | 5 |
| company “Croda”) | |
| Propylene glycol | 5 |
| (L7)₂DDA Powder (as active molecule) | 1 |
| Water | up to 100 |

### EXAMPLE 18: THE COMPOSITION, WHOSE FORMULATION IS GIVEN BELOW IS A HYDROGEL PREPARED AS IN EXAMPLE 14

| | |
|---|---|
| Hydroxyethyl cellulose (sold under the name “Natrosol | 1.5 |
| 250 HR ®” by the company “IMCD”) | |
| Glycerin (sold under the name “Pricerine 9091 ®” by the | 5 |
| company “Croda”) | |
| Propylene glycol | 5 |
| (L7)₂(AF130) Powder (as active molecule) | 1 |
| Water | up to 100 |

### EXAMPLE 19: THE COMPOSITION, WHOSE FORMULATION IS GIVEN BELOW IS A HYDROGEL PREPARED AS IN EXAMPLE 14

| | |
|---|---|
| Hydroxyethyl cellulose (sold under the name “Natrosol | 1.5 |
| 250 HR ®” by the company “IMCD”) | |
| Ethanol | 5 |
| Glycerin (sold under the name “Pricerine 9091 ®” by the | 5 |
| company “Croda”) | |
| Propylene glycol | 5 |
| DDA dilactate Powder (as active molecule) | 1 |
| Water | up to 100 |

### EXAMPLE 20: THE COMPOSITION, WHOSE FORMULATION IS GIVEN BELOW IS AN EMULSION PREPARED AS FOLLOWS

Preparation of aqueous phase A:

| | |
|---|---|
| Hydroxyethyl cellulose (sold under the name “Natrosol | 1.5 |
| 250 HR ®” by the company “IMCD”) | |
| Water | up to 100 |

Preparation of oil phase B:

| | |
|---|---|
| Paraffin oil | 7.5 |
| Dicaprylyl carbonate (sold under the name “Cetiol CC ®” | 4 |
| by the company “BASF”) | |
| Coco caprylate (sold under the name “Cetiol C5 ®” by the | 4 |
| company “BASF”) | |
| Sterate sorbitan and sucrose cocoate (sold the name under | 3 |
| the name “Arlacel 2121 ®” by the company “CRODA”) | |

### Preparation of Phase C:

| | |
|---|---|
| DDA dilactate powder (as active molecule) | 0.5 |
| Water | up to 100 |

Phase B was heated at 80° C. and phase A at the same temperature. Phase B was slowly introduced while under fast stirring into phase A. The mixture was homogenized and cooled under slow stirring: phase C was added when the temperature was below 40° C.

### EXAMPLE 21: THE COMPOSITION, WHOSE FORMULATION IS GIVEN BELOW, IS AN EMULSION PREPARED AS FOLLOWS

Preparation of aqueous phase A:

| | |
|---|---|
| Hydroxyethyl cellulose (sold under the name “Natrosol | 1.5 |
| 250 HR ®” by the company “IMCD”) | |
| Water | up to 100 |

Preparation of oil phase B:

| | |
|---|---|
| Vegetable oil (sold under the name “Cegesoft PS6 ®” by | 5 |
| the company “BASF”) | |
| Dicaprylyl carbonate (sold under the name “Cetiol CC ®” | 3 |
| by the company “BASF”) | |
| Coco caprylate (sold under the name “Cetiol C5 ®” by the | 5 |
| company “BASF”) | |
| Cocoglycerides (sold under the name “Myritol 331 ®” by | 3 |
| the company “BASF”) | |
| Isoceteth-3- Acetate (sold under the name “Hetester | 8 |
| PHA ®” by the company “SACI-CFPA”) | |

### Preparation of Phase C:

| | |
|---|---|
| DDA dilactate powder (as active molecule) | 1 or 0.1 |
| Water | up to 100 |

Phase B was heated at 70° C. and phase A at the same temperature. Phase B was slowly introduced while under fast stirring into phase A. The mixture was homogenized and cooled under slow stirring. Phase C was added when the temperature was below 40° C.

### EXAMPLE 22: THE COMPOSITION, WHOSE FORMULATION IS GIVEN BELOW, IS A DRY OIL PREPARED AS FOLLOWS

Preparation of phase A:

| | |
|---|---|
| Propylene glycol | 5 |
| Dicaprylyl carbonate (sold under the name “Cetiol CC ®” | 34.5 |
| by the company “BASF”) | |
| Coco caprylate (sold under the name “Cetiol C5 ®” by the | 30 |
| company “BASF”) | |
| Propylheptyl Caprylate 30 (sold under the name “Cetiol | 5 |
| Sensoft ®” by the company “BASF”) | |
| Cocoglycerides (sold under the name “Myritol 331 ®” by | 10 |
| the company “BASF”) | |
| Vegetable oil (sold under the name “Cegesoft PS6 ®” by | 10 |
| the company “BASF”) | |
| Passiflora incarnata (sold under the name “Cegesoft | 2 |
| PFO ®” by the company “BASF”) | |

Preparation of phase B:

| | |
|---|---|
| Ethanol | 4.5 |
| basic DDA Powder (as active molecule) | 1 |

Phase B was introduced into phase A under stirring and homogenized.

In all cases corresponding to Examples 12-22, the skin samples show a tanned, uniform and durable natural coloration. The self-tanning effect intensities are obtained by the Fontana-Masson coloration of histological sections and are summarized in the table below.

| | | Self-tanning |
|---|---|---|
| Examples | | effect |
| 1% by weight DDA | Aqueous solution | + |
| in H₂O | | |
| UV (20 mJ/cm²) | / | + |
| 12 | Hydroalcoholic | + |
| | gel | |
| 13 | Hydroalcoholic | + |
| | gel | |
| 14 | Hydrogel | + |
| 15 | Hydrogel | ++ |
| 16 | Hydrogel | ++ |
| 17 | Hydrogel | +++ |
| 18 | Hydrogel | +++ |
| 19 | Hydrogel | ++ |
| 20 | Emulsion | + |
| 21 | Emulsion | + |
| 22 | Dry oil | ++ |

1.-20. (canceled)

21. Appropriately applicable cosmetic composition to ensure tanning of the skin of a human subject, with or without irradiation with ultraviolet rays, containing, in a cosmetically acceptable vehicle, an aqueous solution of at least one compound of formula (I): 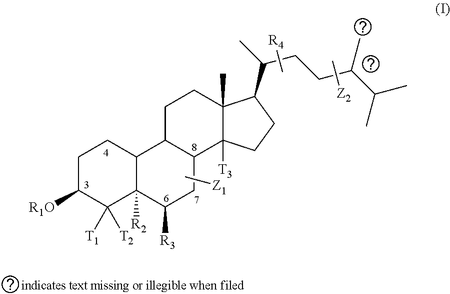
wherein
R₁ designates a hydrogen atom or R₁₀—CO, where R₁₀ represents H, CH₃ or C₂H₅,
R₂ represents hydrogen or OH,
R₃ represents X—(CH₂)ₙ—Y, where n is an integer from 1 and 4, X represents S, O or NH, and Y represents NH₂, imidazol-5-yl, indol-3-yl, piperidin-2-yl, piperidin-3-yl, piperidyn-4-yl, piperazin-2-yl, piperazin-3-yl, pyridin-2-yl, pyridin-3-yl or pyridin-4-yl,
R₄ represents hydrogen or OH in position 20, 22, 24, 25, 26 or 27 of the ring shown above in formula (I), wherein OH is positioned so as to obtain an R or S asymmetric centre,
Z₁ and Z₂ represent the possibility of having single or double bonds between carbons C7 and C8, on the one hand, and carbons C22 and C23, on the other hand,
T₁, T₂, T₃ are, independently from one another, H or CH₃ in α or β, T₄ is H, CH₃ and/or C₂H₅ positioned to form an R or S asymmetric centre, characterized in that it contains an ionic solution of catanionic mixture complexes, where each molecule is formed by a molecule of formula (I) and one or two molecules of a carrier T which is a bolaform surfactant derived from sugar comprising an acid function within its structure and which is obtained by at least one acid-base reaction carried out between the acid group of a carrier (T) and an amino group of substituent R₃ of the carbon in position 6 of the compound of formula (I).

22. Composition according to claim 21, characterized in that it contains a mixture of both compounds of formula (I) wherein R₁=R₄=T₁=T₂=T₃=H, R₂=OH, R₃=NH—(CH₂)₂-imidazol-5-yl and T₄=CH₃ or C₂H₅.

23. Composition according to claim 21, characterized in that it contains 0.01 to 500 g/L of compound(s) of formula (I).

24. Composition according to claim 21, characterized in that it contains at least one tanning compound other than compound(s) of formula (I).

25. Composition according to claim 24, characterized in that the tanning compound(s) it contains, independently from the compound(s) of formula (I), is dihydroxyacetone and/or erythrulose and/or at least one water-soluble dye.

26. Composition according to claim 21, characterized in that it contains at least one melanogenesis activator compound other than the compound(s) of formula (I) selected from the group consisting of substrates for melanin biosynthesis and biological activators for melanogenesis able to act by stimulating melanin synthesis and/or the transfer of melanosomes from melanocytes to keratinocytes.

27. Composition according to claim 26, characterized in that the melanogenesis activator it contains, independently from the compound(s) of formula (I), is chosen from the group consisting of L-tyrosine or derivatives thereof such as N-acetyl-L-tyrosine, L-dihydrophenylalanine, aliphatic diols such as propylene glycol, cyclic diols, adenosine-1 receptor agonists, adenosine-2 receptor agonists, α-hydroxyacids and derivatives thereof, β-hyroxyacids and derivatives thereof, retinoids and derivatives thereof, pro-opiomelanocortic peptides, α-MSH or its analogs, MC1R receptor agonists, cAMP analogs, psoralens, PAR-2 receptor activity activators.

28. Composition according to claim 21, characterized in that it is applied to the skin in an amount between 0.0001 and 100 mg/cm²/day of compound(s) of formula (I).

29. Composition according to claim 21, characterized in that the carrier (T) acid function is selected from the group made up of carboxylic acid COOH, sulfuric acid O—SO₃H, sulfonic acid SO₃H, phosphoric acid O—P(O)(R₅O)OH, wherein R₅ is a C₁-C₆ alkyl group, phosphonic acid O—P(O)R₆OH and phosphinic acid P(O)R₆OH, wherein R₆ is H or a C₁-C₆ alkyl group.

30. Composition according to claim 21, characterized in that the sugar derivative surfactant is a monosaccharide or polysaccharide derivative.

31. Composition according to claim 30, characterized in that the sugar derivative is a glucose or lactose derivative.

32. Composition according to claim 21, characterized in that the sugar acid derivative comprises a sugar structure on which a linear or branched aliphatic chain (CH₂)ₚ is bonded by an NH group, of which the end opposite to the sugar structure carries an acid group, p being an integer having a value from 4 to 10.

33. Composition according to claim 21, characterized in that the ratio of the weight amounts A of compound(s) of formula (I) and B of carrier(s) (T) is between 0.001 and 1000.

34. Composition according to claim 21, characterized in that it contains from 0.01 to 150 g of the totality of components A and B per litre of composition.

35. Composition according to claim 21, characterized in that it contains at least one adjuvant selected from the group consisting of at least one thickener and/or water-soluble dye and/or fragrance and/or alcohol and/or vitamin and/or emulsifier, used alone or in mixture with optionally a co-emulsifier and/or oil or another fat and/or preservative and/or antioxidant and/or bactericide.

36. Composition according to claim 21, characterized in that it contains at least one chemical or physical sun filter, or a mixture of such filters.

37. Composition according to claim 21, characterized in that it is packaged as nano capsules or nanosized vesicles suspended in a liquid medium.

38. Composition according to claim 21, characterized in that it is packaged in the form of hydrogels, creams, lotions, emulsions or aerosols.
